(19) United States
(12) Patent Application Publication
(10) Pub. No.: US 2011/0144669 A1
(43) Pub. Date: Jun. 16, 2011

(54) ANEURYSM COVER DEVICE FOR EMBOLIC DELIVERY AND RETENTION

(75) Inventors: Frank P. Becking, Santa Clara, CA (US); Arturo Rosqueta, San Jose, CA (US); Martin S. Dieck, Campbell, CA (US); Maria Aboytes, Palo Alto, CA (US); Nicholas C. Debeer, Montana, CA (US); Leigh Firestone, Berkeley, CA (US)

(73) Assignee: Nfocus Neuromedical Inc.

(21) Appl. No.: 12/677,269

(22) PCT Filed: Sep. 11, 2008
(86) PCT No.: PCT/US08/76083

§ 371 Date: Jan. 20, 2011

Related U.S. Application Data
(60) Provisional application No. 60/971,573, filed on Sep. 11, 2007.

(51) Int. Cl. A61B 17/08

(52) U.S. Cl. 606/158

(57) ABSTRACT

An implant for treating brain aneurysms, especially terminal aneurysms, comprises a neck cover and elongate shaft removably secured to an embolic delivery catheter. As such, the shaft aids in directing and placing the cover at the aneurysm neck, protecting the delivery catheter from adhesion with the embolic material, and securing the cover in place with connection or adhesion of the shaft to the embolic material delivered through the catheter. The implant can be anchored at the aneurysm either by interface and/or adhesion of the shaft or shaft and cover with the resident embolic materials.

### BACKGROUND OF THE INVENTION

Numerous treatment strategies and devices have been devised to better treat brain aneurysms located at vessel bifurcation locations. These and other terminal aneurysms often grow large and have wide necks due to the direct path of blood pounding into the vascular malformation.

In effort to more effectively treat such aneurysms, U.S. Pat. No. 6,344,048 to Chin discloses a temporary cover made of braid that is manipulated at attachment points on each end of the braid to expand and cover the neck of an aneurysm while embolic material (liquid or coils) are delivered thereto. U.S. Pat. No. 6,746,468 to Septka at FIG. 56 described another temporary cover to assist in holding coils or liquid embolic within terminal aneurysms.

Neither device can function (or be reasonably modified to function) as a permanent implant. Their stated purpose and function is merely temporary in nature, and the potential utility of an implant suitable for coordinated use with embolic delivery has not been appreciated. The present invention offers such utility, and more as will be appreciated upon review of the subject disclosure.

### SUMMARY OF THE INVENTION

The invention is a device for bridging the neck of either a wide-necked or narrow-necked aneurysm in the vasculature and stabilizing the presence of vaso-occlusive or other embolic filler substances. The embolic filler substances can include, e.g. (Trufill™ n-BCA supplied by Cordis, cyanoacrylates, such as those provided by Tenaxis Medical, Inc, including polyethelene glycol (PEG) and derivative compositions, Onyx™—provided by EV3, Inc. or about 50-75% NBCA & Ethiodol). Any of the substances can include additional agents in powder or particulate form. For example, to improve visualization the substances can additionally include agents such as Platinum, Tantalum or metal particles (e.g., for fluoroscopic visualization). The filler substances and the agents can be in any one of several forms including, e.g. gel, suspension, liquid, or semi-liquid forms.

In addition, the devices and methods described herein can be used with other conventional aneurysm filler bodies such as embolic coils (e.g., platinum detachable, polymer or another configuration of coil), biological, biodegradable, or bioabsorble materials such as microfibrillar collagen, various polymeric beads and polyvinylalcohol foam. The polymeric agents may additionally be crosslinked, sometimes in-vivo, to extend the persistence of the agent at the vascular site or increase its ability to promote a desired biological response in the aneurysm such as embolization or endotheliazation.

In a general sense, the invention includes positioning a braid implant having an integral cover and elongate shaft at the neck/entrance and within an aneurysm, respectively. In use, the elongate shaft or securing section of the implant is positioned within the aneurismal sack, with the cover element positioned at the neck of the aneurysm, either just inside the neck or just outside the neck. Generally, the cover will most preferably abut the opening or neck of the aneurysm. Upon embolic material delivery, the implant shaft stabilizes and secures the cover at the aneurysm by contact with the embolic material even after the implant (cover and shaft) is separated from the embolic delivery catheter.

The elongate shaft and cover of the implant are most optimally integral units, both formed from one section of braid. The implant can be derived from a braid tube, and thus is most optimally configured having two layers of braid so that the proximal hub of the cover presents no loose ends. Formation of each of the shaft and cover portion is accomplished by heat setting wire braid with complimentary forms or other tooling that set the braid to the shape desired for each of the cover and the shaft.

The elongate shaft facilitates appropriate positioning of the cover at the neck by using the embolic delivery catheter that is placed within the shaft as a guide for positioning the cover connected to the shaft (actually, in the case of a preferred briad construction—integreally formed). This feature is particularly useful for addressing coverage of wide-neck aneurysms, and aneurysms having irregular shaped openings in which placement may be all the more difficult—especially win an unstablized implant.

The embolic delivery catheter/implant core member is then used to deliver a fill material to treat the aneurysm and capture at least the shaft/securing section of the implant within the aneurysm. A part of the embolic delivery catheter can remain in the elongate shaft when the proximal portion of the catheter is detached therefrom, or the entire catheter can be removed after the delivery of the embolic material.

Capture and retention of the implant at/within the aneurysm may be by adhesion of the filler material with the implant. The capture can also be by physical interlocking with the lattice defined by the braid/matrix of the shaft or cover (or both)—or a combination of interlocking and adhesion.

The cover portion of the implant may be used to retain the embolic material. On the other hand, the aneurysm may be filled only so much as necessary to secure the implant. In which case, the density of the cover and the flow disruption effect it offers, whether placed inside or outside the neck of the aneurysm, can be curative. In such cases, the braid must have sufficient density (e.g., somewhat as pictured) to offer relevant flow-disruption properties.

Even if not intended for use as a flow disrupter, a relatively tighter braid matrix in the cover may offer an excellent matrix for tissue growth. When fully endothelialized across the neck, the aneurysm is cured. The subject implant can help promote such outcome due to the known tendency of adequately tight wire braid surfaces to promote proximal tissue endothelization.

Employing a braid of sufficient wire count to offer density for flow disruption and/or endotheliazation also provides some gross structural benefits. Namely, with higher wire counts in the cover (e.g., by employing one or more layers adding up to about 96 wire count, and more preferably about 144, 192 or higher, the cover takes on the shape of a substantially circular periphery (whether set flat as a disc or cupped in shape). As compared to structures having lesser wire counts that merely resemble flower petals, the full circular periphery provides both a better barrier to embolic extravasation, and a continuous/uniform fit with curvilinear vascular anatomy in opposition thereto. The clear benefits imparted to the treatment of the aneurysm by this configuration are better overall aneurysm seal and/or more complete endothelization across the neck of the aneurysm into adjacent tissue.

The present invention includes the braid implant device alone (including the cover and shaft) as well as a system that includes the implant in combination with the position-fixing filler (i.e., the embolic material). The entire medical device may also be defined as the implant (including the integral braid cover and elongate shaft) together with the embolic delivery catheter positioned within the elongate shaft. The catheter is removed after the embolic material is delivered. In some embodiments, at least a portion of the catheter can be retained within the shaft of the implant.

In some variations, the invention includes an implant and embolic delivery catheter configured to function as noted above, wherein the embolic delivery catheter is releasably retained within the shaft by a slip fit. A slightly tighter loose interference fit may, likewise, be employed. In either case, an abutment feature will be provided at the distal end of the implant shaft (e.g., a platinum marker band) so the delivery catheter can function effectively as a pusher during implant placement.

In other variations, the catheter is releasably retained by a mechanically, electrically or otherwise releasable mounting system. In one example, a threaded interface is provided in which a helical wire or ribbon is affixed (or integral) with the distal outer section of the embolic delivery catheter. This treading is received by the braid, which may be held compressed by an outer sheath or otherwise stabilized to enable a threaded interface between the implant braid and the catheter.

Alternatively, a braid surface may be provided on the distal exterior portion of the embolic delivery catheter. It may be embedded therein and/or secured at its ends. Such a surface will provide a braid-to-braid interface with the shaft of the catheter to offer Velcro™-like interference between the elements. Such an interface will offer a greater degree of control between the elements than a slip fit, but can still be released by simply withdrawing the embolic delivery catheter once the implant is secured in the aneurysm by the material delivered thereto.

Regarding the overall delivery system, it typically comprises a first/outer catheter that contains the compressed braided implant (braided cover and braided shaft) for endovascular delivery, and a second catheter that is an embolic delivery catheter. The embolic delivery catheter is positioned within the elongate shaft of the implant. This catheter may comprise hypotube or be of more typical polymeric (including braid-reinforced) polymer catheter construction.

The first catheter may first be positioned adjacent an aneurysm using convention endovascular access techniques, and the implant and embolic delivery catheter tracked therethough. Alternatively, the first/outer catheter may be preloaded with the embolic delivery catheter such that the implant situated at or adjacent the first/outer catheters distal end and the whole complex advanced simply in an over-the-wire arrangement. Or the system may be adapted for “Rapid-Exchange” delivery to the treatment site.

Systems for treating aneurysms include the implant mounted on the catheter that delivers the embolic materials, and the embolic materials that are delivered. Methods of treating aneurysms include positioning the implant mounted over the embolic delivery catheter at the aneurysm, and delivering the embolic materials. Removal of the delivery catheter may be accomplished by simple withdrawal or by breaking of an interface with the braid, such as provided with threads on the catheter so that it can be unscrewed from the elongate shaft.

### BRIEF DESCRIPTION OF THE FIGURES

Variation of the invention from the embodiments pictured is contemplated. Accordingly, depiction of aspects and elements of the invention in the figures is not intended to limit the scope of the invention, although the figures may serve as antecedent basis for elements in the claims.

FIG. 1 shows the subject implant, outside the outer delivery catheter positioned upon the embolic delivery catheter with the cover adjacent the neck of an aneurysm;

FIG. 2 illustrates filling of the aneurysm with embolic material;

FIG. 3 shows the neck-cover and shaft member retained by and retaining embolic material, with the embolic delivery removed;

FIGS. 4A-4C shows one variation of the subject implant, alone, from various angles; and

FIGS. 5A and 5B detail pre-and-post delivery configurations of inventive system components.

### DETAILED DESCRIPTION

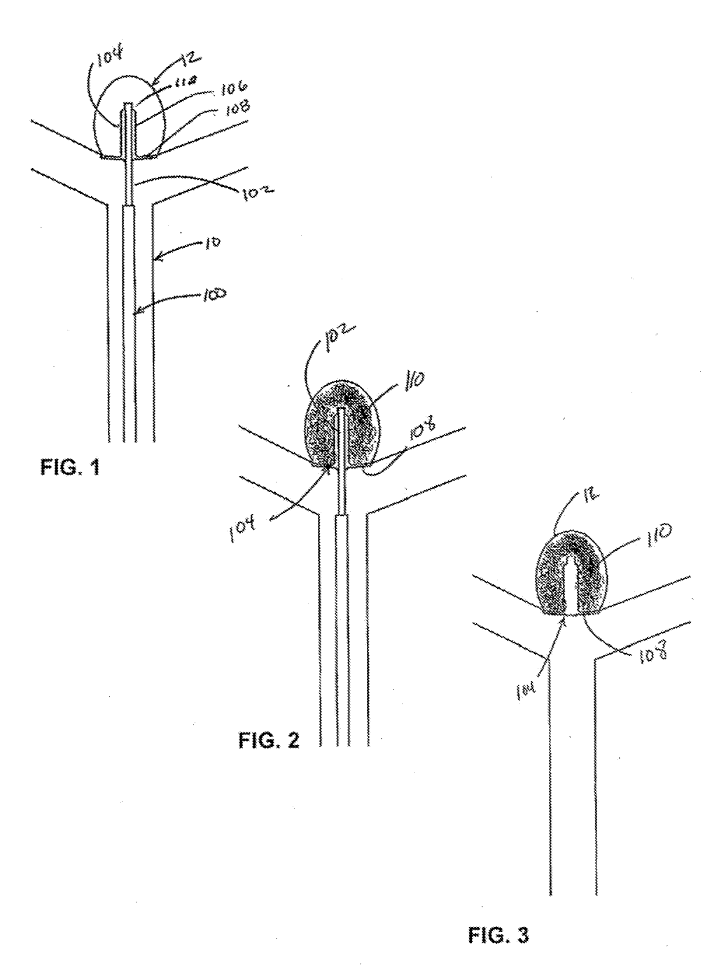

Turning now to FIG. 1 a catheter 100 is advanced within the vasculature 10 to the site of an aneurysm 12. Typically, a catheter or a microcatheter is initially steered into or adjacent to the entrance of an aneurysm, often aided by the use of a steerable guidewire. The wire is then withdrawn from the microcatheter lumen to allow delivery of the subject implant and/or system

A distal end of a core member 102 is located within the entrance of the aneurysm 12. The core member includes a lumen (not shown) and implant 104 (e.g. the combination of the braided shaft and braided cover) releasably set or mounted thereon. Naturally, delivery or guide catheter 100 can be positioned within the aneurysm 12 and then withdrawn while leaving the core member 102 and implant 104 within the aneurysm 12. Alternatively, the implant 104 and core member 102 can be advanced from catheter 100 into the aneurysm.

In variations where catheter 100 is retracted to expose the implant, the implant could later be advanced to help push-off or separate the implant from the catheter (specifically, after the implant is secured with the filler/anchoring material as described further below).

Implant 104 comprises a shaft 106 and cap 108. FIGS. 1-3 illustrate one mode of use in which the neck of the aneurysm is covered alone the luminal side of the vasculature. In another variation, the cap 108 of the implant 104 can both be deployed inside the aneurysm 12, and then snugged-back in a proximal direction to make a seal between inner walls of the aneurysm and the cap 108. Generally, however, the cap 108 is oversized relative to the aneurysm neck and pushed into apposition with the vessel 10 into a saddle shape, then anchored by the vaso-occlusive or other filler/embolic substances described herein. When oversized and pulled to the withdrawn in the aneurysm neck is will assume more of a cup shape. In either case, the neck of the aneurysm is at least substantially covered by the implant and/or the cover helps define a new neck of the aneurysm in more fusiform aneurysm examples.

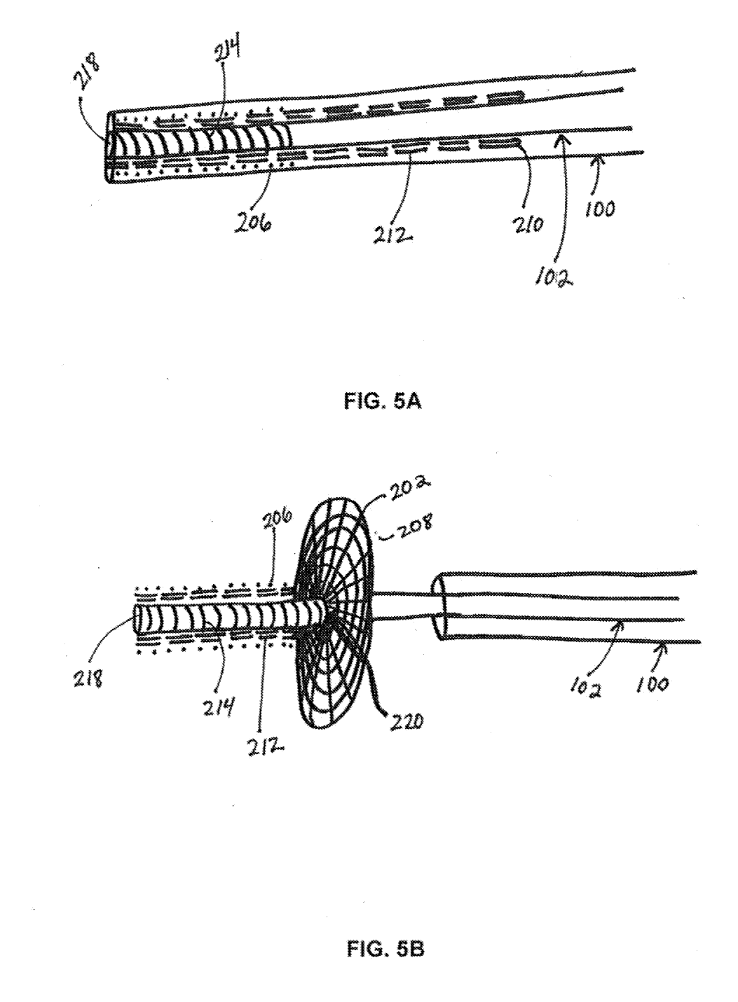

Implant 104 may effectively “plug” the aneurysm. In one construction, the implant is a braid configuration with a double-layer bottom. As detailed further in connection with FIGS. 5A and 5B, the double-layer is advantageously constructed from doubled-over braid, thereby yielding four layers of material in/defining the cap/cover section of the device.

The implant can be crimped by a marker band or affixed to a band with a shoulder, so that the implant 104 is held in a compressed section 112 at the distal end of the core 102 that acts as a pusher for positioning the implant. Implant 104 expands into shape upon exit (by advancement or withdrawal) of the catheter 100, optionally as shown in FIG. 1.

Where there is a shoulder defined in or connection with a band (glued, welded, soldered in place, etc) or otherwise—such as by as stent-like ring—the shaft proximal to that implant/core delivery catheter region may have a relatively expanded profile. Such a shape may offer additional protection from inadvertently capturing core member 102 within the implant “plug” when removal is desired.

The diameter or profile of the cap 108 as well as the length or profile of the shaft 106 can both be optimized either in combination or separately for a range of aneurysm sizes. Generally aneurysm openings range in size from about 4 mm up to as large as 15 mm. Typical sizes are from about 6 mm to about 10 mm.

As shown in FIG. 2, an embolic filler material 110 (as variously described herein) is delivered through the core member 102 (or through another catheter or core member/liner positioned within the core member). The presence of the cap 108 acting a cover helps avoid over-filling the aneurysm and having filler leakage into the vasculature. Also, the embolic filler becomes engaged with the braided shaft 106 to capture the shaft within the aneurysm 12. The embolic filler may adhere to the walls or sac of the aneurysm, or it may simply interfere with the typical less-than-regular morphology present. As noted herein, the delivery device (catheter 100 and core 102) are not captured in this fashion. The density of the cap 108 prevents the embolic filler material 110 from escaping into the vessel. To further insure proper retention of the filler material, the cap can be oversized relative to the aneurysm neck and/or the viscosity of the embolic filler can be adjusted—the latter, especially to accommodate larger pore sizes in the cap.

As shown in FIG. 3, as the implant 104 plugs the aneurysm and is captured by the setting or set embolic filler the delivery system (catheter 100 and/or core member 102) is withdrawn. This withdrawal allows the cap 108 to fully close. In one variation, the braid of the cap (with its highest density at the cap closure) stagnates the flow of the embolic material in the open chamber. In some cases, the cap promotes growth of tissue about the neck of the aneurysm to aid in retention of the implant 104.

Its one variation, the catheter or core member is breakable or detachable proximal to the tip. (Stated otherwise, embolic delivery catheter 102 may include a breakable/detachable tip.) For example, catheter 102 could have a rubber/polymer sleeve holding sections of the catheter at a butt-joint proximal to the distal end of the core member. To detach the implant, the catheter is simply pulled once the implant is captured by the filler/embolic substances. Alternatively, the release mechanism can include a GDC-type erodable joint. In another variation, the joint may be a mechanical detachment structure having a micro nut and screw mechanism or the catheter may incorporate an outer screw helix that interfaces with the implant shaft. However, any such arrangement could be provided. Furthermore, the “joint” could be located within the expandable braid “shaft” section of the implant or even proximal to the entire implant and cap. However, it would be best if any residual catheter core is held or set inside the implant braid shaft, so that nothing hangs down from the implant. In any case, examples of potentially suitable detachment structures are found in U.S. Pat. Nos. 5,261,916 to Engelson; 5,250,071 to Palermo; 5,122,136 and 5,354,295, each to Guglielmi et al.—the entirety of each of the above patents are incorporated by reference.

As shown in FIG. 1, the implant 104 forms an extra-sacular cover (vs. and endo-sacular approach where an implant is contained within the aneurysm.) The endovascular approach offers an improved chance of complete aneurysm neck coverage. This feature may be very desirable since not all aneurysm necks/openings are round, or oriented along the axis of the axial vascular approach to a bifurcation aneurysm as pictured. Also, by apposition with healthy tissue of the vessel the cap 108 provides a natural platform extension for tissue endotheliazation.

Promoting endothelialization in this manner can help further capture the device at the aneurysm, but also may yield a faster path to a fully-healed/reformed neck. Moreover having a cap 108 larger than the aneurysm neck/opening and in the vasculature (vs. inside the aneurysm sac) can offer further stability for implant positioning as well as resisting the so-called “water hammer” effect of blood pounding at the site of a terminal aneurysm. While such features are especially useful at terminal aneurysms (e.g., at vessel bifurcations), the device can also be used at a side-wall aneurysm.

From a mechanical perspective, while the braid forming the implant advantageously comprises Nitinol (NiTi) alloy that is superelastic at body temperature, filaments within the braid could be bioabsorbable, resorbable, and/or erodible filaments. Such filaments could include magnesium; PLA/PGLA, Polycarbonate, etc. In one example, the braid is made from woven cables (typically twisted cable) material in which one or more of the members of the cable is erodible/resorbable. Some or all of the cable used to make-up the braid may incorporate a polymer (e.g., PGLA) or a metal such as Magnesium for a “disappearing” element(s), together with primarily structural material elements such as Stainless Steel, PT, PTW, TaW, Ti, NiTi, NiTiNb, CoCro, etc. This approach allows for a greater measure of tissue incorporation of the overall implant, without hindering mechanical performance (e.g., navigation, delivery, etc.).

In addition, the embolic filler material (or the implant) may comprise one or more drug-carrying polymer members. Suitable compositions include the J&J Cypher™ coating applied by vascular devices manufactured by Surmodics, or that used by Biosensors where the coating is in filamentous form. The drug may any drug capable of promoting a desired biological activity in the aneurysm or proximal to it. Accordingly, the drug could be, e.g. sirolimus, an analog or derivative thereof, or another effective antiproliferative macrocyclic triene, a drug promoting endotheliazation, etc.

Note also, the same implant may include more than one adjunctive element. For example the implant may contain drug-loaded polymer members and also have a feature that imparts tissue-growth promoting properties (such as a selected porosity or wire surface treatment, including a simple black oxide—vs. highly polished—finish) to achieve a highly-tuned biological response.

When different metals in a braided coil are used, those intended to remain unaffected by the presence of dissimilar metal (the “structural” part of the cable) may be paralene coated to avoid galvanic/bi-metallic corrosion. Or such material can be left bare to accelerate dissolution of the more reactive (less noble) metal, allowing the Mg (or another resorbable/erodible metal or alloy) to operate as a sacrificial anode.

The length of the shaft 106 as well as size of the cap 108 of the implant 104 can be variable to account for different aneurysm geometries/morphologies (tall and big ones vs. small and short ones). Generally, the shaft braid portion 106 will have a length between about 2 mm and about 7.5 mm. (i.e., typically at least about 0.1 inches in length) to provide a stable or otherwise adequate interface between the implant and the catheter that pushes/carries it. An internal diameter (ID) if the shaft may be between about 0.01 inches to about 0.05 inches to accommodate variously sized core members for embolic material delivery. The cap 108 should have a radius between about 2 mm and about 8 mm (diameter about 4 mm to about 15 mm). At the lower end of the range, the cover will be able to fully open and deploy in the parent vasculature, in larger sizes, the cap/cover will typically be for intra-anerusymal use. Certain ratios of the cover to shaft may provide improved results to obtain good shaft capture by the embolic material given the diameter required to treat a given aneurysm. For example, such improvements may be obtained with ratios between about 1:3 and about 1:1 (ratio expressed as shaft length:cap diameter). Likewise, the length of the shaft section offers advantages in terms of directing the connected cap in view of the stable attachment to the manipulable core delivery catheter 102.

The end of the catheter 100 or the core member 102 may terminate at or beyond the distal end of the implant 104. A longer tip extension (not shown) ensures filling the dome of the aneurysm first, where the embolic filler would then continue downward toward the cap 108.

However, such an arrangement does not offer the advantage of the core catheter 102 being fully “hidden” behind braid to avoid its capture. Furthermore, such an arrangement may only be appropriate with the proximal-release improvements described above and/or with liquid filler material that does not capture the catheter or core member. Naturally, lubricious, hydrophilic or other coatings may be applied to the catheter/core to help avoid trapping the catheter or core member. In any case it may be advantageous to hydrophilically coat the delivery catheter sheath to assist in system navigation to the implantation site.

As discussed above, when the embolic filler agent sticks to tissue, very little of the aneurysm might be filled and the plug still be captured. When the embolic does not bind to tissue, filling more (or all) of the aneurysm may be necessary to ensure good capture of the implant. In which case, the irregular aneurysm geometry will “lock” the mass in place, with sections of the material penetrating the braid of the implant, thereby securing at least that part of the device.

To ensure such lock-up, the braid structure of the shaft may be more open/porous than the cap (to ensure filler penetration). Also, having a denser cap allows the cap to serve as an effective cover for the aneurysm itself (if not backed by embolic material as shown in the figures). Note that a different/varying pitch to the braid may be used for such purposes. Or, the effect may be achieved by using double-layer braid (or more) used along the cap. Still, the device could be single-layer throughout, with different or variable braid pitch. The wire count of the braid can be in a range from 24 to 144 wires, most optimally in a range from about 24 or 32 wires to about 72 wires in various multiple of layers, in wire size typically ranging from about 0.0008″ to about 0.00125″. Still, other options are possible to achieve various objectives.

The shape of the braid architecture may be formed in a number of ways, for example, setting a NiTi braid, at a particular temperature as understood by those with skill in the art. Still, Ti or Steel or another material may be plastically deformed (or annealed into such a shape) with the same ultimate effect as heat-setting the NiTi.

In a general sense, the invention includes positioning an implant with a shaft or securing section within an aneurysm sack, while positioning a cover element outside the sack, to abut or otherwise span the opening/adjacent of the aneurysm opening. Then, fill material is delivered into the aneurysm to at least partially embolize the volume and also capture at least the shaft/securing section within the aneurysm. The capture of the implant to the aneurysm may be by adhesion of the filler material with the implant. The capture can also be by physical interlocking with the lattice defined by the braid/matrix—or a combination of both.

The above method can be modified where only the shaft is captured within the aneurysm leaving a space between the filler and cap. In such a case, the cap has sufficient density to serve to enable blood stagnation and thrombus formation. Alternatively, the above method can include positioning the implant so that the shaft or capturing section as well as the cap are in contact with the filler.

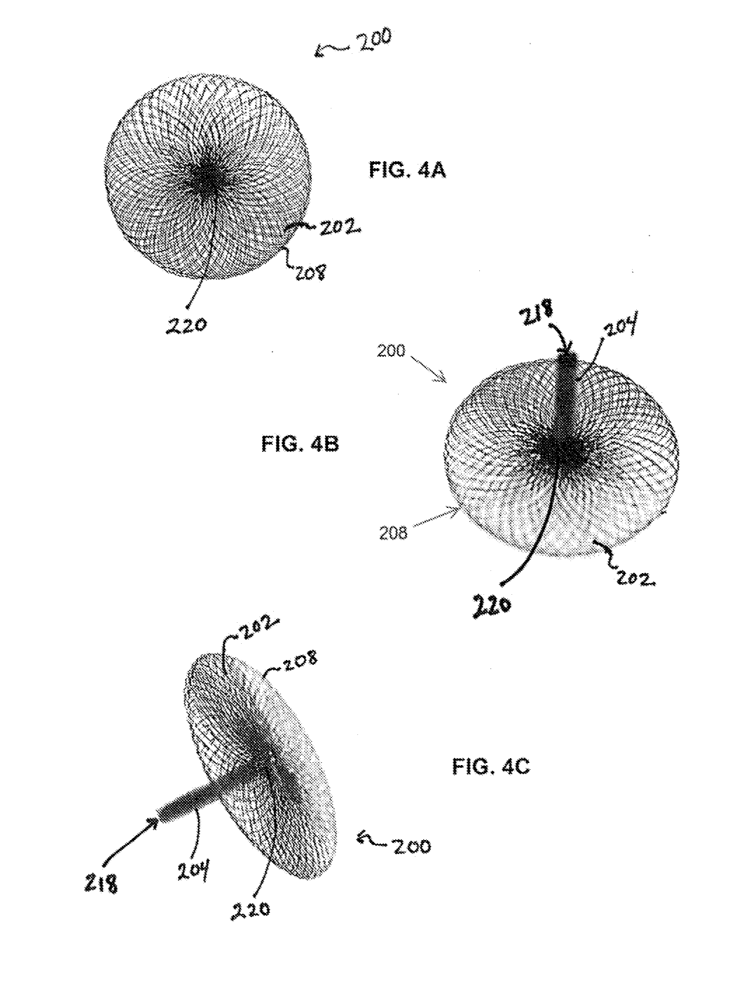

A prototype implant 200 that is particularly suitable for such use is pictured from different angles in FIGS. 4A-4C. In these views, elements of implant 200 include a braided cover 202 having a substantially circular rim 208 formed from folded tubular braid, and a consolidated braid shaft 204 contiguously formed base junction 220 integrally connecting cover 202 and shaft 204. Braid shaft 204 is captured by a tubular heat shrink 206 into a set-diameter bundle with interior lumen 218 provided for retention of catheter 102. Ata proximal closure 210 of the braid it inverts or folds back on itself as shown. Consequently, no loose ware ends are present in this region of the device, ends are generated from the configuration of the braid implant.

The specific construction and operation of implant 200 shown in FIGS. 4A-4C is diagrammatically illustrated in FIGS. 5A and 5B. Here, a doubled-back/over section of braid 212 is shown. The proximal end comprises bends or turns 210 which form the circular rim 208 of the cover 202 upon deployment of the device.

The result of this architecture for the cover and shaft yields four layers of material in/defining the cover section 202 of the device.

The shaft portion 204 is shown covered by heat shrink 206 to define a diameter allowing for screw thread interaction between a helix 214 on the embolic delivery core member catheter 102 and the braid 212 itself.

The recovery action of the device (viewing 5A to 5B) illustrates cover 202 formation from the tubular braid 212. This recovery may be by SMA recovery, elastic or superelastic action as noted above.

The invention includes methods of treatment as well as the implant device alone and the implant in combination with the position-fixing filler. In additional variations, the invention includes an implant configured to function as noted above, where the implant is mounted to a delivery system. However, the delivery system may simply be a catheter and sheath type system where each is a commercially available unit such as an off-the-shelf microcatheter and a larger off-the-shelf microcatheter or guide catheter. The embolic delivery catheter 102 carrying the implant merely requires a lumen of such size to deliver embolic filler therethough to the aneurysm; and the larger microcatheter/guide catheter 100 allow the implant/core member 102/104 construct pass therethrough. The system components can be advanced in an over-the-wire arrangement, or the system could be adapted for “Rapid-Exchange” use/utility.

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

The subject methods may include each of the physician activities associated with implant positioning and release. As such, methodology implicit to the positioning and deployment of an implant device forms part of the invention.

Also, it is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a” “an” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language. All references cited are incorporated by reference in their entirety. Although the foregoing invention has been described in detail for purposes of clarity of understanding, it is contemplated that certain modifications may be practiced within the scope of the appended claims.

What is claimed is:

1. A medical device assembly for treating an aneurysm comprising:
a delivery catheter adapted to deliver embolic material to an aneurysm, and
an implant comprising braided wire including an aneurysm neck cover and an elongate shaft releasably retained upon a distal end of the catheter.

2. The assembly of claim 1, wherein the wire comprises superelastic NiTi.

3. The assembly of claim, wherein the cover comprises two layers of braid.

4. The assembly of claim 3, wherein the cover comprises four layers of braid and at least a portion of the shaft also comprises two layers of braid.

5. The assembly of claim 1, wherein the shaft has an interior diameter (ID) between about 0.01 inches to about 0.05 inches.

6. The assembly of claim 1, wherein the shaft has a length of at least about 0.1 inches.

7. The assembly of claim 1, wherein the elongate shaft is adapted to direct positioning of the cover across the neck of the aneurysm.

8. The assembly of claim 1, wherein the elongate shaft is releasably retained upon the catheter by an interference fit.

9. The assembly of claim 8, where the interference fit is provided between threading on the catheter distal end and the braid of the elongate shaft.

10. The assembly of claim 1, wherein the cover has a substantially circular rim.

11. The assembly of claim 1, wherein the cover has sufficient density to substantially disrupt blood flow into the aneurysm to allow thrombus formation therein.

12. The assembly of claim 1, wherein the shaft is at east substantially perpendicular to the cover.

13. A system for treating an aneurysm comprising:
a medical device assembly according to claim 1; and
liquid embolic material at least partially filling the aneurysm and contacting the shaft, thereby securing the implant with the cover at the neck of the aneurysm upon catheter removal.

14. A method of treating an aneurysm comprising:
positioning a distal end of an embolic delivery catheter within an aneurysm, the distal end releasably retaining an elongate shaft of an implant to position a cover portion of the implant at a neck of the aneurysm;
delivering liquid embolic material through the delivery catheter into the aneurysm, the liquid embolic securing the implant position along an elongate shaft of the implant, while the shaft protects the catheter from also being secured by liquid embolic; and
removing the embolic delivery catheter.

15. The method of claim 14, wherein the removing is accomplished by unscrewing a threaded interface between the catheter and the implant.

16. The method of claim 14, wherein the liquid embolic is delivered until contacting the cover, the cover substantially preventing the liquid embolic from exiting the aneurysm.

17. The method of claim 4, wherein the aneurysm is a terminal aneurysm.
